(19) United States
(12) Patent Application Publication
(10) Pub. No.: US 2009/0143724 A1
(43) Pub. Date: Jun. 4, 2009

(54) Microprojection Array Immunization Patch and Method

(75) Inventors: Michel J.N. Cormier, Mountain View, CA (US); James A. Matriano, Mountain View, CA (US); Peter E. Daddona, Menlo Park, CA (US); Juanita A. Johnson, Belmont, CA (US); Wendy A. Young, Cupertino, CA (US)

Correspondence Address: Edwards Angell Palmer & Dodge LLP, P.O. Box 55874, Boston, MA, 02205, US

(73) Assignee: Alza Corporation, Mountain View, CA (US)

(21) Appl. No.: 12/367,318

(22) Filed: Feb. 6, 2009

Related U.S. Application Data
(63) Continuation of application No. 11/267,563, filed on Nov. 4, 2005, now abandoned.
Application No. 11/267,563 is a continuation of application No. 10/127,171, filed on Apr. 20, 2002, now abandoned.
(60) Provisional application No. 60/285,572, filed on Apr. 20, 2001.
(60) Provisional application No. 60/342,552, filed on Dec. 20, 2001.

(51) Int. Cl. A61M 37/00

(52) U.S. Cl. 604/47

(57) ABSTRACT

Skin patches (20) having a microprojection array (10), a reservoir (18) containing an antigenic agent and an immune response augmenting adjuvant, and methods of using same to vaccinate animals (e.g., humans) is disclosed. In a preferred embodiment, the microprojection arrays (10) are composed of a photoetched and micro-punched titanium foil (14). The microprojections (12) are coated with a liquid formulation containing a vaccine antigen and an adjuvant such as glucosaminyl muramyl dipeptide, dried, and applied to skin of the animal to be vaccinated using an impact applicator. The microprojections (12) create superficial pathways through the stratum corneum to facilitate permeation of antigenic agent and adjuvant. Antigen dose and depth of penetration can be controlled. This technology has broad applicability for a wide variety of therapeutic vaccines to improve efficacy, and convenience of use.

### CROSS REFERENCE TO RELATED APPLICATIONS

Priority is claimed from U.S. Patent Applications Ser. Nos. 60/285,572 filed Apr. 20, 2001 and 60/342,552 filed Dec. 20, 2001.

### BACKGROUND ART

Vaccination can be achieved through various routes of administration, including oral, nasal, intramuscular (IM), subcutaneous (SC), and intradermal (ID). It is well documented that the route of administration can impact the type of immune response. See LeClerc, et al. “Antibody Response to a Foreign Epitope Expressed at the Surface of Recombinant Bacteria: Importance of the Route of Immunization,” Vaccine, 1989. 7: pp 242-248.

The majority of commercial vaccines are administered by IM or SC routes. In almost all cases, they are administered by conventional injection with a syringe and needle, although high velocity liquid jet-injectors have had some success. See for example Parent du Chatelet et al, Vaccine, Vol. 15, pp 449-458 (1997).

In recent years, a growing interest in the development of needle-free vaccine delivery systems has emerged. Independent laboratories have demonstrated needle-free immunization to macromolecules, including protein- and DNA-based antigens. Glenn et al. demonstrated that a solution containing tetanus toxoid mixed with an adjuvant, cholera toxin, applied on untreated skin is capable of inducing anti-cholera toxin antibodies. Glenn et al, Nature, Vol. 391, pp 851 (1998). Tang et al, demonstrated that topical administration of an adenoviral vector encoding human carcinoembryonic antigen induces antigen-specific antibodies. Tang et al., Nature, Vol. 388, pp 729-730 (1997). Fan et al, also demonstrated that topical application of naked DNA encoding for hepatitis B surface antigen can induce cellular and humoral immune responses. Fan et al, Nature Biotechnology, Vol. 17, pp 870-872 (1999).

The skin is a known immune organ. See for example Fichtelius, et al., Int. Arch. Allergy, 1970, Vol. 37, pp 607-620, and Sauder, J. Invest. Dermatol, 1990, Vol. 95, pp 105s-107s. Pathogens entering the skin are confronted with a highly organized and diverse population of specialized cells capable of eliminating microorganisms through a variety of mechanisms. Epidermal Langerhans cells are potent antigen-presenting cells. Lymphocytes and dermal macrophages percolate throughout the dermis. Keratinocytes and Langerhans cells express or can be induced to generate a diverse array of immunologically active compounds. Collectively, these cells orchestrate a complex series of events that ultimately control both innate and specific immune responses. Indeed, exploitation of this organ as a route for immunization has been explored. See for example Tang et al, Nature, 1997, Vol. 388, pp 729-730; Fan et al, Nature Biotechnology, 1999 Vol. 17, pp 870-872; and Bos, J. D., ed Skin Immune System (SIS), Cutaneous Immunology and Clinical Immunodermatology, 2^{nd} Ed., 1997, CRC Press, pp 43-146. A recent publication discusses transdermal vaccination using a patch. See Glenn et al, “Transcutaneous Immunization: A Human Vaccine Delivery Strategy Using a Patch”, Nature Medicine, Vol. 6, No. 12, December 2000, pp 1403-1406. However, to date, a practical, reliable, and minimally invasive method for delivering antigens specifically into the epidermis and/or dermis in humans has not been developed. A significant limitation to intradermal injection with conventional needles requires a very high level of eye-hand coordination and finger dexterity.

The skin's primary barrier, the stratum corneum, is impermeable to hydrophilic and high molecular weight drugs and macromolecules such as proteins, naked DNA, and viral vectors. Consequently, transdermal delivery has been generally limited to the passive delivery of low molecular weight compounds (<500 daltons) with limited hydrophilicity.

A number of approaches have been evaluated in an effort to circumvent the stratum corneum barrier. Chemical permeation enhancers, depilatories, occlusion, and hydration techniques can increase skin permeability to macromolecules. However, these methods may not be able to deliver therapeutic doses without prolonged wearing times, and they can be relatively inefficient means of delivery. Furthermore, at nonirritating concentrations, the effects of chemical permeation enhancers are limited. Physical methods of permeation enhancement have also been evaluated, including sandpaper abrasion, tape stripping, and bifurcated needles. While these techniques increase permeability, it is difficult to predict the magnitude of their effect on drug absorption. Laser ablation, another physical permeation enhancer, may provide more reproducible effects, but it is currently cumbersome and expensive. Active methods of transdermal delivery include iontophoresis, electroporation, sonophoresis (ultrasound), and ballistic delivery of solid drug-containing particles. Delivery systems using active transport (e.g., sonophoresis) are in development, and delivery of macromolecules is possible with such systems. However, at this stage, it is not yet known if these systems will allow successful and reproducible delivery of macromolecules in humans.

Microprojection array patch technology is being developed to increase the number of drugs that can be transdermally delivered through the skin. Upon application, the microprojections create superficial pathways through the transport barrier of the skin (stratum corneum) to facilitate hydrophilic and macromolecule delivery.

### DESCRIPTION OF THE INVENTION

Microprojection arrays having a plurality of stratum corneum-piercing microprojections are used to intradermally deliver an antigenic agent and immune response augmenting adjuvant to induce a potent immune response in mammals, particularly in humans. The immune response augmenting adjuvant is delivered intradermally in an amount which is effective to augment the skin's immune response to the antigenic agent. The use of the adjuvant preferably allows for a lesser amount of antigenic agent delivery while still achieving therapeutically effective antigen antibody titers in the patient i.e., a dose sparing effect.

Preferably, the antigenic agent comprises a vaccine antigen which antigens are typically in the form of proteins, polysaccharides, alegosacarides, lipoproteins and/or weakened or killed viruses. Particularly preferred antigenic agents for use with the present invention include hepatitis virus, pneumonia vaccine, flu vaccine, chicken pox vaccine, small pox vaccine, rabies vaccine, and pertussis vaccine.

The immune response augmenting adjuvant is preferably selected from those materials which are known to augment the mammal's immune response to antigens and which do not promote adverse skin reactions in the patient. Most preferred is Gerbu adjuvant: N-acetygiucosamine-(β1-4)-N-acetylmuramyl-L-alanyl-D-glutamine (GMDP).

The reservoir containing the antigenic agent and the immune response augmenting adjuvant can be a gel material, preferably in the form of a thin film laminated to the microprojection array, but more preferably is a material which is applied as a coating directly onto the microprojections. Most preferably the coating is applied only on the skin piercing tips of the microprojections.

In use, the microprojection array is applied to the skin of an animal to be vaccinated and the array is pressed against the animal's skin causing the microprojections to pierce the outermost layer (i.e., the stratum corneum layer) of the skin. Most preferably, the microprojection array is applied to the skin of an animal to be vaccinated using an applicator which impacts the microprojection array against the skin, causing the microprojections to pierce the skin. For intradermal delivery of the antigenic agent and the adjuvant in accordance with the present invention, the microprojects should pierce through the stratum corneum and into the underlying epidermis and dermis layers of the skin. Preferably, the microprojects do not penetrate the skin to a depth which causes significant bleeding. To avoid bleeding, the microprojections should pierce the skin to a depth of less than about 400 μm, preferably less than about 200 μm. The microprojections create superficial pathways through the stratum corneum to facilitate permeation of the antigenic agent and the adjuvant. Antigen dose and depth of microprojection penetration are easily controlled. This intradermal vaccine and method of vaccinating animals has broad applicability for a wide variety of therapeutic vaccines to improve efficacy, and convenience of use.

### BRIEF DESCRIPTION OF THE DRAWINGS

FIG. 1 is a perspective view of a microprojection array in accordance with the present invention;

FIG. 2 is a perspective view of a microprojection array having a solid antigen-containing coating on the microprojections;

FIG. 3 is a side sectional view of an intradermal antigen delivery device used in Example 1;

FIG. 4 is a graph showing skin penetration depth of the microprojections in animal skin;

FIG. 5 is a graph of ovalbumin delivered versus time for the study performed in Example 1;

FIG. 6 is a graph of ovalbumin-specific antibody (IgG) titers versus time from individual guinea pigs immunized with OVA delivered by the microprojection array, in which the arrows indicate the time of primary and booster immunizations;

FIG. 7 is a graph of ovalbumin-specific antibody (IgG) titers in hairless guinea pigs immunized with OVA comparing microprojection delivery with intradermal, subcutaneous and intramuscular deliveries;

FIG. 8 is a graph of antibody (IgG) titers from guinea pigs immunized with OVA alone, and together with an immune response enhancing adjuvant, comparing delivery via microprojection array and intradermal injection, one week after the booster administration;

FIG. 9 is a graph showing amounts of ovalbumin coated onto microprojection arrays, and delivered into animals over 5 second and 1 hour wearing times, as discussed in detail in Example 2;

FIG. 10 is a graph showing ovalbumin delivery efficiency achieved in the methods described in Example 2;

FIG. 11 is a graph of antibody titers comparing an ovalbumin-coated microprojection array with several doses of ovalbumin administered by intradermal injection; and

FIG. 12 is a graph showing amounts of GMDP and ovalbumin coated onto microprojection arrays, and delivered into animals over various wearing times, as discussed in Example 2.

### MODES FOR CARRYING OUT THE INVENTION

The present invention provides an intradermal vaccine and method for intradermally delivering an antigenic agent and an immune response augmenting adjuvant useful for vaccinating animals. The terms “intradermal”, “intracutaneous”, “intradermally” and “intracutaneously” are used herein to mean that the antigenic agent (e.g., a vaccine antigen) and adjuvant are delivered into the skin, and specifically into the epidermis layer and/or underlying dermis layer of the skin.

The term “microprojections” refers to piercing elements which are adapted to pierce or cut through the stratum corneum into the underlying epidermis layer, or epidermis and dermis layers, of the skin of a living animal, particularly a human. The piercing elements should not pierce the skin to a depth which causes bleeding. Typically the piercing elements have a microprojection length of less than 500 μm, and preferably less than 250 μm. The microprojections typically have a width of about 75 to 500 μm and a thickness of about 5 to 50 μm. The microprojections may be formed in different configurations and/or shapes, such as needles, hollow needles, blades, pins, punches, and combinations thereof.

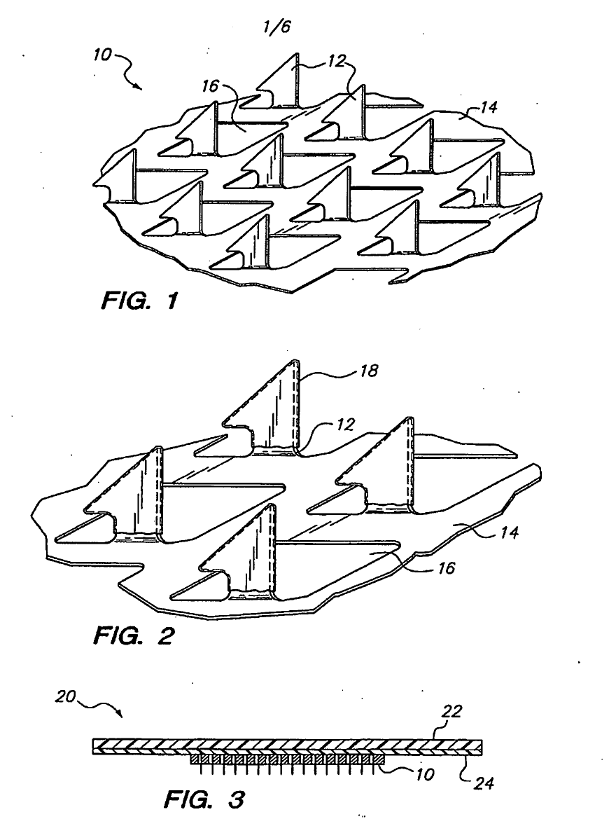

The term “microprojection array” as used herein refers to a plurality of microprojections arranged in an array for piercing the stratum corneum. The microprojection array may be formed by etching or punching a plurality of microprojections from a thin sheet and folding or bending the microprojections out of the plane of the sheet to form a configuration such as that shown in FIG. 1 and in Trautman et al., U.S. Pat. No. 6,083,196. The microprojection array may also be formed in other known manners, such as by forming one or more strips having microprojections along an edge of each of the strip(s) as disclosed in Zuck, U.S. Pat. No. 6,050,988. Other microprojection arrays, and methods of making same, are disclosed in Godshall et al., U.S. Pat. No. 5,879,326 and Kamen, U.S. Pat. No. 5,983,136. The microprojection array may also be in the form of a plurality of hollow needles which hold a dry antigenic agent and adjuvant.

The intradermal vaccine of the present invention includes a microprojection array having a plurality of stratum corneum-piercing microprojections extending therefrom and having a reservoir containing an antigenic agent (e.g., a vaccine antigen) and an immune response augmenting adjuvant. The reservoir is positioned, relative to the microprojections in the microprojection array, so that the reservoir is in antigenic agent-transmitting and adjuvant-transmitting relation to the slits cut through the stratum corneum by the piercing microprojections. In one embodiment, the reservoir can be a material (e.g., a gel material) in the form of a thin polymeric film laminated on the skin proximal or skin distal side of the microprojection array. Reservoirs of this type are disclosed in Theeuwes et al. WO 98/28037, the disclosures of which are incorporated herein by reference. More preferably, the antigenic agent and adjuvant are in a coating applied directly on the microprojections, most preferably on the piercing tips of the microprojections. Suitable microprojection coatings and apparatus useful to apply such coatings are disclosed in U.S. patent application Ser. Nos. 10/045,842 filed Oct. 26, 2001; 10/099,604 filed Mar. 15, 2001; and another application filed concurrently herewith and claiming dependency from U.S. provisional application Ser. No. 60/285,576 filed Apr. 20, 2001, the disclosures of which are incorporated herein by reference. The microprojections are adapted to pierce through the stratum corneum into the underlying epidermis layer, or epidermis and dermis layers, but preferably do not penetrate so deep as to reach the capillary beds and cause significant bleeding. Typically, the microprojections have a length which allows skin penetration to a depth of less than about 400 μm, and preferably less than about 300 μm. Upon piercing the stratum corneum layer of the skin, the antigenic agent and adjuvant contained in the coating are released into the skin for vaccination therapy.

FIG. 1 illustrates one embodiment of stratum corneum-piercing microprojection member 10 for use with the present invention. FIG. 1 shows a portion of the member 10 having a plurality of microprojections 12. The microprojections 12 extend at substantially a 90° angle from a sheet 14 having openings 16. The member 10 may be incorporated in an agent delivery or sampling system 20 (shown in FIG. 3) including a backing 22 and adhesive 24 for adhering the system 20 to the skin. In the embodiment of the microprojection member 10 shown in FIGS. 1, 2 and 3, the microprojections 12 are formed by etching or punching a plurality of microprojections 12 from a thin metal sheet 14 and bending the microprojections 12 out of a plane of the sheet. Metals such as stainless steel and titanium are preferred. Metal microprojection members and methods of making same are disclosed in Trautman et al, U.S. Pat. No. 6,083,196; Zuck U.S. Pat. No. 6,050,988; and Daddona et al., U.S. Pat. No. 6,091,975 the disclosures of which are incorporated herein by reference. Other microprojection members that can be used with the present invention are formed by etching silicon using silicon chip etching techniques or by molding plastic using etched micro-molds. Silicon and plastic microprojection members are disclosed in Godshall et al. U.S. Pat. No. 5,879,326, the disclosures of which are incorporated herein by reference.

FIG. 2 illustrates the microprojection member 10 having microprojections 12 having an antigen-containing coating 18. The coating 18 may partially or completely cover the microprojections 12. The coatings can be applied to the microprojections 12 by dipping the microprojections into a volatile liquid solution or suspension of the protein antigen and optionally any immune response augmenting adjuvant. The liquid solution or suspension should have an antigenic agent concentration of about 1 to 20 wt. %. The volatile liquid can be water, dimethyl sulfoxide, dimethyl formamide, ethanol, isopropyl alcohol and mixtures thereof. Of these, water is most preferred.

Suitable antigenic agents which can be used in the present invention include antigens in the form of proteins, polysaccharides, oligosaccharides, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof. A number of commercially available vaccines which contain antigenic agents may also have utility with the present invention and include flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Suitable immune response augmenting adjuvants which, together with the antigenic agent, can be used in the present invention include aluminum phosphate gel; aluminum hydroxide; algal glucan, β-glucan; cholera toxin B subunit, heat-shock proteins (HSPs); gamma inulin, GMDP (N-acetylglucosamine-(β1-4)-N-acetylmuramyl-L-alanyl-D-glutamine); GTP-GDP; Imiquimod; ImmTher™ (DTP-GDP); Loxoribine, MPL®; MTP-PE; Murametide; Pleuran (β-glucan); Murapalmitine; QS-21; S-28463 (4-Amino-α,α-dimethyl-1H-imidazo[4,5-c]quinoline-1-ethanol); Scalvo Peptide (IL-1β163-171 peptide); and Theramide™.

The microprojection array intradermal vaccine of the present invention is preferably applied to the skin of the patient under impact conditions. For example a biased (e.g., spring driven) impact applicator of the type described in Trautman et al. U.S. patent application Ser. No. 09/976,798 filed Oct. 12, 2001, the disclosures of which are incorporated herein by reference, can be used to apply the coated microprojection arrays of the present invention. Most preferably, the coated microprojection array is applied with an impact of at least 0.05 joules per cm² of the microprojection array in 10 msec or less.

The preferred antigenic agent-containing and adjuvant-containing reservoir useful with the present invention is in the form of a solid coating directly on the surfaces of the microprojections. Preferably, the coating is applied in a liquid state and then dried. The volatile liquid solution or suspension containing the antigenic agent and adjuvant can be applied to the microprojection array by immersion, spraying and/or other known microfluidic dispensing techniques. Thereafter, the coating is allowed to dry to form a solid antigen and adjuvant-containing coating. Preferably, only those portions of the microprojection array which penetrate into the skin tissue are coated with the antigenic agent. Suitable microprojection coating methods and apparatus are disclosed in Trautman et al. U.S. patent application Ser. No. 10/099,604 filed Mar. 15, 2002, the disclosures of which are incorporated herein by reference. Using the coating methods disclosed therein and the coating compositions disclosed herein, we have been able to precisely and uniformly coat only the tips of the skin piercing microprojections in typical metal (i.e., titanium) microprojection arrays having microprojection lengths of less than 500 μm.

While the relative amounts of adjuvant and antigenic agent delivered intradermally in accordance with the present invention will vary depending upon the particular antigenic agent and adjuvant being delivered, typically the weight ratio of delivered adjuvant to delivered antigen should be in the range of about 0:5 to 50:1 and more preferably in the range of about 1:1 to 10:1. In order to achieve these adjuvant-to-antigenic-agent delivery ratios, the reservoir preferably contains loadings of the antigenic agent and the immune response augmenting adjuvant in the same weight ratios disclosed immediately above.

Furthermore, with microprojection tip coating, antigenic agent and adjuvant loadings of at least 0.2 μg per cm² of the microprojection array, and preferably at least 2 μg per cm² of the array are easily achieved. For a typical 5 cm² array, this translates into antigenic agent and adjuvant loadings of at least 1 μg, and preferably at least 10 μg, which is more than adequate for most vaccinations. With microprojection tip coating of the antigenic agent and adjuvant, the delivery efficiency (E_{del}) is greatly enhanced. E_{del} is defined as the percent, by weight, of the antigenic agent and adjuvant released from the coating per predetermined period of time. With tip coating of the antigenic agent and adjuvant-containing solutions or suspensions, an E_{del} of at least 30% in 1 hour, and preferably at least 50% in 15 minutes can be achieved. Thus, the present invention offers significant cost advantages over conventional macrotine skin piercing devices used in the prior art.

In the following examples, the depth of microprojection skin penetration, model antigen (i.e., OVA) delivery, and the ability of the intradermally delivered model antigen to provoke an immune response, were evaluated in guinea pigs. In these experiments, the microprojections penetrated the skin to an average depth of about 100 μm. Different doses of OVA were obtained by varying the coating solution concentration, wearing time, and system size. With a 2 cm² microprojection array, 1 to 80 μg of OVA was delivered, and a delivery rate as high as 20 μg in 5 seconds was achieved. Dose-dependent primary and secondary antigen-specific antibody responses were induced. At 1 and 5 μg doses, the antibody response was equivalent to that observed after intradermal administration and up to 50-fold greater than that observed after subcutaneous of intramuscular administration. A solid coating of the adjuvant, GMDP, with OVA resulted in augmented antibody responses. Thus, microprojection array patch technology allows intracutaneous administration of dry antigens.

Control of intracutaneous OVA delivery by the microprojection array was achieved by varying the concentration of the coating solution, wearing time, and system size, and the combination of these variables allows for greater flexibility in the dosage. These results are also applicable to other protein antigens. Moreover, because most compounds are more stable in a dry state, microprojection array technology has the potential to eliminate cold-chain storage.

The microprojection array system was well tolerated in the guinea pigs. The mild and transitory application-site erythema after primary immunization is consistent with the shallow penetration of the microprojections into the skin. Following booster administration with the microprojection array or ID injection, the moderate erythema and edema suggests a mixed immunologic response.

### EXAMPLE 1

The immunization studies had two objectives: to measure the immune response caused by delivery of varying amounts of OVA from microprojection arrays in hairless guinea pigs (HGPs), and to compare the results against immunization with the microprojection array using a low level of OVA together with the GMDP adjuvant. Outbred male and female euthymic HGP were obtained from Biological Research Labs (Switzerland, strain ibm:GOHI-hr) and Charles River Labs (Michigan, strain IAF:HA-HO-hr). Animals were 250 to 1000 grams. Animals were quarantined, individually housed, and maintained in a facility accredited by the Association for Assessment and Accreditation of Laboratory Animal Care. The research adhered to the Principles of Laboratory Animal Care (NIH publication #85-23, revised 1985).

The microprojection arrays used in these studies had 330 μm projections at a density of 190 microprojections/cm² over a 1 or 2 cm² area. The microprojection arrays were produced using controlled manufacturing processes incorporating an autoCAD-generated microprojection array design, photochemical etching, and forming. First, a thin laminate resist was applied on a sheet of titanium about 30 μm thick. The resist was contact-exposed using a mask with the desired pattern and developed using a process very similar to that used in the manufacture of printed circuit boards. The developed sheet was then acid etched, and the microprojections were bent at an angle of about 90° relative to the plane of the sheet using a forming tool. The finished microprojection array was a screen with precision microprojections as shown in FIG. 1.

The microprojection arrays were coated with ovalbumin (OVA) and glucosaminyl muramyl dipeptide (GMDP) or with only OVA as a control. For the studies using GMDP (Pharmitra, United Kingdom) the microprojection arrays were immersed in a solution containing OVA (1%) and GMDP (10%). For the comparison studies using OVA alone the arrays were coated with OVA by immersion in 1%, 5%, or 20% OVA (Grade V, SIGMA Chemical Co, St Louis, Mo.) in sterile water. Excess solution was removed by forced air and the arrays were air-dried for 1 or more hours at room temperature. For the studies that used fluorescein isothiocyanate (FITC)-labeled OVA (Molecular Probes, Portland, Oreg.), the fluorescent compound alone was used for any coating solution containing 5% OVA or less. For OVA coating solutions at 20%, unlabeled OVA (15%) was mixed with FITC-OVA (5%).

The amount of OVA coated on the microprojection arrays was determined using FITC-OVA. The dry OVA coated on the device was extracted by immersing the device in 10 mL boric acid (0.1 M, pH 9) for 1 hour at room temperature in a glass scintillation vial. An aliquot of the extracted material was further diluted in boric acid for quantitation against known standards by luminescence spectrometry (excitation 494 nm, emission 520 nm). Microprojection arrays coated with FITC-OVA were also inspected visually by fluorescence microscopy.

Following coating and drying, the microprojection arrays were affixed to low-density polyethylene backings with a polyisobutylene adhesive. The final systems had a structure as shown in FIG. 3 and a total surface area of 8 cm² and the arrays had a skin contact area of either 1 cm² or 2 cm².

The treatment sites (lateral area of the thorax) of anesthetized HGPs were cleaned with isopropyl alcohol wipes (70%) and allowed to dry. The skin site was lightly stretched manually when the system was applied using an impact applicator. Following application, the stretching tension was released and the system was left on the skin for the specified period of time. For devices left on skin for more than 5 seconds, the HGPs were wrapped with Vetwrap® (3M, St Paul, Minn.) and individually housed.

To evaluate the depth of microprojection penetration, the system was removed immediately after application and the skin site was dyed with a cotton swab imbibed with India ink. The dye was applied in a circular motion in two opposing directions for approximately 15 seconds. The excess dye was then wiped off with gauze, and isopropyl alcohol wipes were used to remove any dye from the skin, until only the pathways created by the microprojection array were visible. Subsequently, the HGPs were euthanized and the skin sites removed and frozen. Each frozen skin site was biopsied with one 8-mm biopsy punch. Biopsies were sectioned parallel to the skin surface, with the first section at 20 μm and the remainder at 50 μm. Then the individual skin sections were mounted on microscope slides, and the dyed holes in each slice were counted. From these data and from the known density of microprojections, the percentage of pathways that were dyed in a particular skin section was calculated and plotted as a function of depth. In some studies, skin sites were photographed using a video microscope system (Hi-Scope KH2200, Hirox Co, Japan).

Each HGP received a dry-coated FITC-OVA microprojection array, which was applied as described above. Following system removal, the treated skin sites were thoroughly washed with 70% isopropyl alcohol to remove any residual OVA on the skin surface. The HGPs were euthanized and 8-mm skin biopsies were taken. Each tissue sample was placed in a scintillation vial with 0.1 mL deionized water. Hyamine hydroxide (0.9 mL, 1 M in methanol, JT Baker, Phillipsburg, N.J.) was then added, and the samples were incubated overnight at 60° C. Thereafter, the dissolved material was further diluted with 2 mL hyamine hydroxide/water (9:1), and fluorescence was quantitated by fluorometry and compared to known standards. Background control samples included untreated skin. A minimum replicate of three was used for each experimental condition.

Baseline blood samples were obtained from all animals before the day of immunization. On the day of immunization, the HGPs were anesthetized and the treatment sites were cleaned with 70% isopropyl alcohol and allowed to dry. For immunizations performed by needle injection, OVA was dissolved in sterile water. Sterile 1-mL syringes with 25-gauge needles (Becton Dickinson, Franklin Lakes, N.J.) were used. ID and SC injections were performed on the dorsal-lateral area of HGPs. The quadriceps muscle of the hind leg was used for IM injection. Microprojection arrays containing dry-coated OVA were applied as described above.

Each HGP received a primary immunization (Day 0) followed by a secondary (i.e., booster) immunization 4 weeks later with an identical article. After primary immunization, HGPs were anesthetized and blood was collected from the anterior vena cava. The serum samples were evaluated by immunoassay for the presence of anti-OVA antibodies.

Sera from nonimmunized and immunized HGPs were tested for the presence of antibodies to OVA by enzyme-linked immunosorbent assay (ELISA). Briefly, 96-well polystyrene plates (Maxisorp, NUNC, Rochester, N.Y.) were coated with 0.1 muwell of OVA (10 μg/mL in 0.2 M Na bicarbonate/carbonate buffer, pH 9.6) and incubated overnight at 4° C. The plates were washed with PBS-Tween buffer then blocked with 200 μL of PBS/casein (0.5%)/Tween-20 (0.05%) buffer for 1 hour at room temperature. Then the plates were again washed and the test sera were added (100 μL/well at 2- to 5-fold serial dilutions, three replicates, 1 hour at room temperature). After washing, 100 μL peroxidase conjugated goat anti-guinea pig IgG antibody (Jackson ImmunoResearch Laboratories, West Grove, Pa.) was added and incubated for 1 hour at room temperature. After incubation, the plates were washed, 100 μL of substrate (ABTS, Becton Dickinson, Franklin Lakes, N.J.) was added, and they were incubated for 35 minutes at room temperature. Absorbance (4051490 nm) was measured using a SpectraMAX 250 (Molecular Devices Corporation, Sunnyvale, Calif.). The results are expressed as endpoint antibody titers relative to nonimmunized control sera samples.

Results are presented as the mean with its associated standard error of the mean. Comparison between groups was performed by analysis of variance (ANOVA).

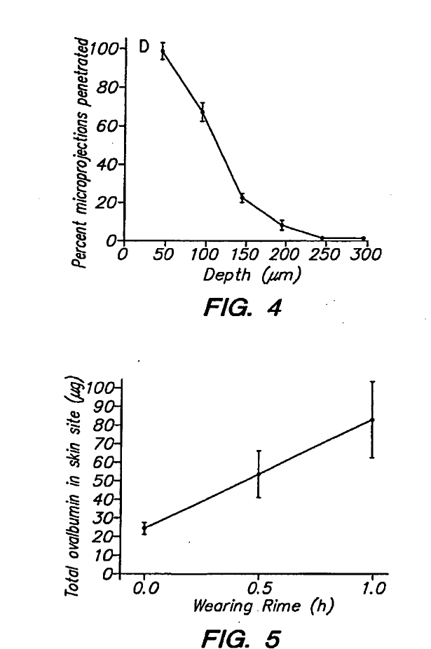

The microprojection array patches were applied to HGP and were visually assessed for signs of skin erythema, edema, and bleeding. When compared to untreated skin no detectable erythema to mild reactions were generally observed after the application process. Any erythema that did develop was transient, typically resolving within 24 hours or less. No signs of edema or bleeding were evident. Evaluation of the microprojection penetration using the India ink technique, showed that >95% of the microprojections penetrated through the stratum corneum barrier. Moreover, a relatively uniform penetration pattern was observed. Skin biopsies taken from treated sites revealed that approximately 50% of the microprojections penetrated to the depth of about 100 μm (FIG. 4). No microprojection penetrated deeper than 300 μm.

Increasing the concentration of OVA in the coating solution resulted in increased loading of OVA on the microprojection arrays. With a 1% OVA coating solution, the amount of OVA coated was approximately 7 μg/cm². Microprojection arrays coated with a 5% OVA coating solution contained about 40 μg/cm² dry-coated OVA, and those coated with a 20% OVA coating solution contained about 240 μg/cm² dry-coated OVA (Table 1). Observation by fluorescence microscopy revealed that the coating was present as a thin amorphous glass. At the maximum concentration, the average calculated thickness was about 3 μm, which was consistent with the microscopic observations OVA delivery from 2 cm² microprojection arrays coated with the three OVA concentrations was evaluated with systems applied on HGP skin for 5 seconds. These studies found that 1%, 5%, and 20% OVA coating solutions resulted in the delivery of an average of about 1, 6, and 10 μg/cm² of protein, respectively (Table 1).

TABLE 1
Amount of Ovalbumin Coated on Microprojection Arrays and
Delivered into Hairless Guinea Pig Skin^{a}

| | Amount of ovalbumin | |
|---|---|---|
| | coated on | Amount of ovalbumin |
| Ovalbumin coating | microprojection array | delivered |
| Concentration (%) | (μg/cm²; mean ± SEM) | (μg/cm²; mean ± SEM) |
| 1 | 7.4 ± 0.6 | 0.9 ± 0.1 |
| 5 | 42.2 ± 1.9 | 5.8 ± 1.4 |
| 20 | 238 ± 20 | 9.9 ± 0.6 |

Microprojection patch arrays (2 cm²) were coated with fluorescein isothiocyanate (FITC)-labeled ovalbumin. Arrays were applied on hairless guinea pigs (n=3) for 5 seconds.

Using a 2 cm² device coated with a 20% OVA solution, the delivery of protein into the skin increased with longer application times (FIG. 5). A 5 second application delivered approximately 20 μg of OVA into the skin. A 30 minute application delivered 50 μg of OVA, and a 1 hour application delivered approximately 80 μg. The results indicate a linear relationship as a function of time versus amount delivered.

Immunization studies were conducted to determine whether delivery of OVA from microprojection arrays could induce an immune response in HGPs. Animals were divided into four treatment groups (n=3 to 5/group) receiving 1, 5, 20, or 80 μg of OVA/group, as established by the delivery studies. Table 2 summarizes the OVA coating concentration, patch wearing time, and device surface area used to deliver the approximate doses of antigen.

TABLE 2
Ovalbumin Delivery in Hairless Guinea Pig Skin from
Ovalbumin-Coated Microprojection Arrays

Delivery condition

| | I | II | III | IV |
|---|---|---|---|---|
| Ovalbumin coating concentration (%) | 1 | 5 | 20 | 20 |
| Wearing time (seconds) | 5 | 5 | 5 | 3600 |
| Surface area (cm²) | 1 | 1 | 2 | 2 |
| Approximate dose delivered (μg) | 1 | 5 | 20 | 80 |

Each HGP received a primary immunization. Four weeks thereafter, a booster immunization was performed under identical priming conditions. To determine the level of OVA-specific antibody (IgG) titers by ELISA, serum was collected from each animal at weekly intervals.

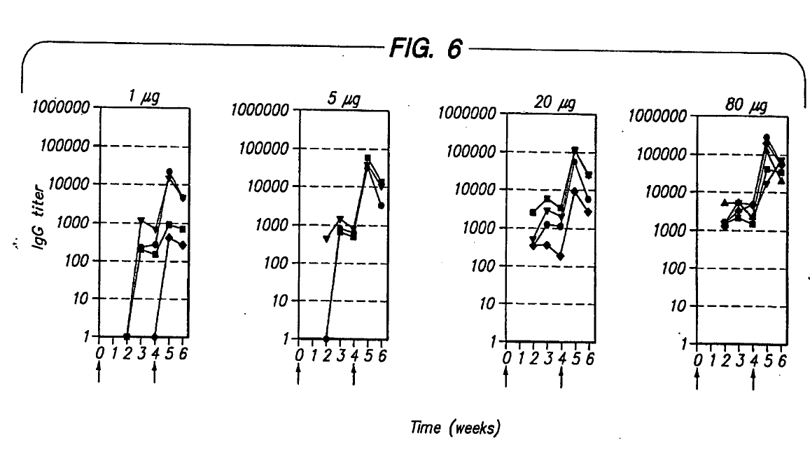

The immune response of each HGP to 1, 5, 20 and 80 μg of OVA delivered by microprojection array is shown in FIG. 6. Relatively low levels of OVA-specific antibodies were observed 2 weeks after the primary immunization. Over the next 4 weeks, a general increase in antibody titer was observed. The seroconversion rates increased with increasing antigen dose and with increasing time. All animals that received 20 or 80 μg doses of OVA seroconverted by 2 weeks after the primary immunization. All animals had seroconverted after the booster immunization at all doses tested. A dramatic increase in antibody titer was observed 1 week after booster administration. In general, peak antibody titers were observed 1 week following the booster immunization. Thereafter, antibody titers decreased until the next booster treatment was administered.

Additional studies were conducted to compare immunization with the microprojection array to conventional ID, SC, and IM injections. The doses of OVA tested were 1, 5, 20, and 80 μg. Serum samples taken after the primary immunization demonstrate that the kinetics of the antibody response to OVA using needle administration was similar to that observed using the microprojection array. In all treatment groups, an increase in the OVA dose resulted in an increase in OVA-specific antibody titers. Higher antigen doses correlated with increased seroconversion rates after primary immunization (data not shown). With the exception of a few animals immunized with low doses of OVA (i.e., SC at 1 μg, IM at 1 and 5 μl), all other HGPs had detectable anti-OVA antibodies 2 weeks after the booster immunization.

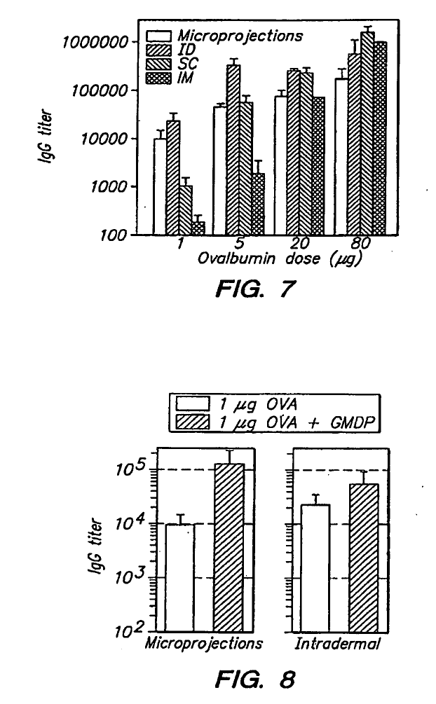

ANOVA was performed to evaluate possible differences among the various treatment groups, analyzing antibody titers 1 week after the booster immunization (FIG. 7). A significant dose-response effect was observed for all methods of antigen delivery. Animals immunized with 20 or 80 μg of OVA using the microprojection array had antibody titers comparable to those immunized by conventional ID, SC, or IM injection. Animals receiving 5 μg of OVA via the microprojection array had significantly greater (24 fold) antibody titers than those seen with IM needle administration. A 1 μg dose of OVA delivered by the microprojection array resulted in higher antibody levels compared to the SC (10 fold) or IM (50 fold) injection routes.

Studies were conducted to determine whether an adjuvant co-formulated with OVA and dry-coated onto the microprojection array could enhance the antibody responses. Immunization studies using microprojection arrays dry-coated with OVA and GMDP, delivered approximately 1 μg of OVA along with 15 μg GMDP, and resulted in a significant increase in antibody titers over non-adjuvant controls. Following ID administration, the increase in antibody titer was 250%. Following microprojection array administration, the increase in antibody titer was 1300% (FIG. 8).

The antibody response following delivery of a low antigen dose (1 μg) could be enhanced by co-delivery of the adjuvant GMDP. Delivery studies with OVA and GMDP dry-coated arrays demonstrated that the presence of the adjuvant did not significantly affect the amount of OVA delivered (data not shown). Although the amount of GMDP delivered into the skin using the microprojection array could not be directly quantified, we estimated that about 15 μg of GMDP was delivered into the skin based on mass transfer calculations. At this dose, GMDP boosted the antibody response in both ID and microprojection arrays routes of administration but the effect was significantly greater following microprojection array co-administration of GMDP and OVA. In addition, the antibody titers generated with microprojection arrays that delivered GMDP and OVA approached the titer levels achieved with OVA doses of 20 μg or greater in the absence of GMDP, which demonstrates a significant dose-sparing effect. The difference in enhancement observed between microprojection array delivery and ID is not understood at this time but may be the result of subtle differences in antigen and adjuvant localization in the different layers of the skin following ID or microprojection array administration. Indeed, experiments have demonstrated that OVA localizes primarily in the epidermal layers following microprojection array delivery (data not shown). Such a preferred localization may result in increase exposure of relevant epidermal cells, such as Langerhans cells, to the adjuvant, which may trigger enhanced activation.

The microprojection arrays were well tolerated in the HGP. Following primary immunization, erythema at the application site was mild and dissipated within 24 hours. In addition, no signs of infection were observed in any of the animals. Following booster administration with the microprojection array or ID injection, moderate skin erythema and edema was observed. This skin reaction appeared rapidly and lasted a few days, suggesting a mixed immunologic response.

The skin is rich in antigen-presenting cells and skin-associated lymphoid tissue, making it an ideal target for immunization. Indeed, a number of studies have demonstrated that ID or epicutaneous administration of antigens leads to effective immune responses and a dose-sparing effect compared to other routes of administration. However, a significant limitation of conventional ID administration is the difficulty in precisely controlling the depth of penetration, requiring skilled personnel. Our results demonstrate that OVA coated on microprojection arrays can be delivered intracutaneously in a reproducible manner. Moreover, specific immunity was induced following OVA delivery by microprojection array. Both primary and secondary antigen-specific antibody responses were generated using dry antigen coated on the microprojection arrays. The response was dose dependent. The kinetics of the antibody response towards OVA administered with the microprojection array systems was similar to that observed using conventional injection. Microprojection administration at 1 and 5 μg doses gave immune responses up to 50-fold higher than that observed following the same subcutaneous or intramuscular dose. Dry coating an adjuvant, glucosaminyl muramyl dipeptide, with OVA on the microprojections resulted in augmented antibody responses.

### EXAMPLE 2

An aqueous solution containing 20 wt % ovalbumin was prepared. The ovalbumin was tagged with FITC for subsequent analysis. Microprojection arrays (microprojection length 250 μm, 595 microprojections per array) had an area of 2 cm². The tips of the microprojections were coated with this solution by passing the arrays over a rotating drum carrying the OVA solution using the apparatus and method disclosed in co-pending U.S. patent application Ser. No. 10/099,604 filed Mar. 15, 2002. On some arrays, multiple coatings were performed. Fluorescence microscopy revealed that in all cases, the coating was limited to the first 100 μm of the microprojection tip. Quantitation by fluorimetry demonstrated that 1.8 μg, 3.7 μg, and 4.3 μg were coated on the arrays following 1, 2, and 4 coatings, respectively.

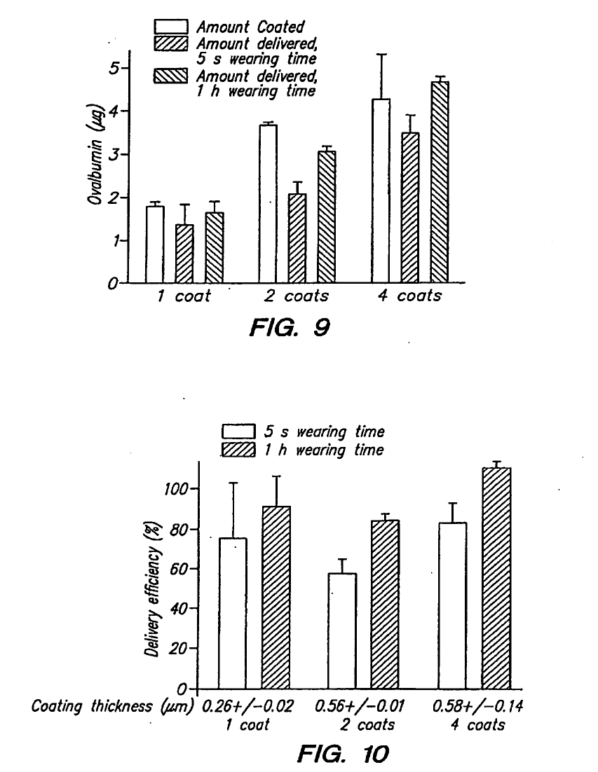

Some of these microprojection arrays were applied to hairless guinea pigs (three animals per group) for evaluation of ovalbumin delivery into the skin. The skin of the animal flank was stretched manually bilaterally ( [symbol] and [symbol] ) at the time of application of the system. Application was performed with an impact applicator (total energy=0.4 Joules, delivered in less than 10 milliseconds) using a spring-driven impact applicator of the type disclosed in U.S. patent application Ser. No. 09/976,798 filed Oct. 12, 2001. The system applied comprised an ovalbumin coated microprojection array, adhered to the center of a low density polyethylene film backing with an acrylate adhesive (7 cm² disc). Following application, the stretching tension was released and the system was removed after 5 seconds or 1 hour contact with the skin. Following removal of the system, residual drug was thoroughly washed from the skin and an 8 mm skin biopsy was taken at the location of the application. The total amount of ovalbumin delivered in the skin was determined by dissolving the skin biopsy in hyamine hydroxide (1M in methanol). Quantitation was performed by fluorimetry. Results, presented in FIGS. 9 and 10, demonstrate that up to 4.5 μg of OVA can be delivered into hairless guinea pig skin with delivery efficiency higher than 55 and 85% following a 5 second and 1 hour wearing times, respectively. Delivery efficiency was also found to be relatively independent of the thickness of the coating.

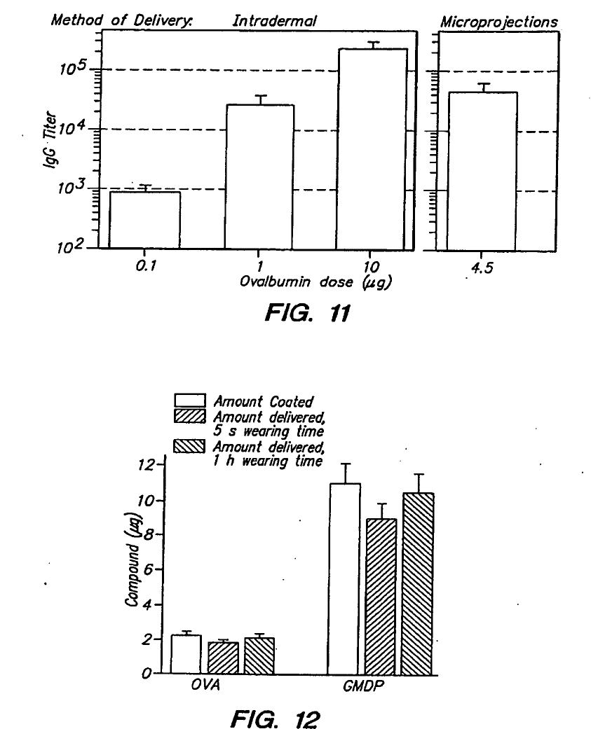

Identical microprojection arrays were coated with untagged ovalbumin using a similar methodology. The amount of protein coated on the arrays was evaluated by total protein assay. The target dose of 5 μg of ovalbumin (OVA) was coated with acceptable reproducibility (4.6±0.5 μg) using a 20 wt % OVA coating solution. Immunization studies were conducted with these arrays in one group of six hairless guinea pigs. Systems and system application in animals was the same as described above except that the wearing time in all guinea pigs was 5 seconds. Three additional groups of animals received intradermal injections of 0.1, 1.0, and 10 μg ovalbumin. Blood samples were taken at various time intervals and evaluated for antibody (IgG) titer against ovalbumin by ELISA. Two and three weeks after primary immunization, all animals dosed with the microprojection array patch had developed anti-ovalbumin IgG antibodies, demonstrating that antigen tip-coated microprojection arrays are effective in inducing an immune response (see FIG. 11). A dose response was observed with increasing doses of ovalbumin administered intradermally. Extrapolations from this dose response demonstrated that the antibody response obtained with the microprojection arrays was consistent with an intradermal delivery of about 1.5 to 4 μg ovalbumin.

Experiments similar to those described above are performed using an aqueous coating solution containing 2 wt % ovalbumin and 10 wt % GMDP. Eight coatings are performed per array. GMDP coated and delivered into the skin is estimated from the amount of ovalbumin coated and delivered and the ratio of GMDP to ovalbumin in the coating formulation. Analysis reveals that each microprojection array is coated with 11 μg GMDP and 2.2 μg ovalbumin. Scanning electron microscopy examination reveals that the coating is present as a glassy amorphous matrix with good uniformity of coating from microprojection to microprojection. The coating is limited to the first 150 μm of the microprojection. Delivery studies in the hairless guinea pig indicate that GMDP is delivered with a delivery efficiency similar to that of ovalbumin (FIG. 12).

The microprojection array patch of the present invention is broadly applicable to intracutaneous delivery of a wide variety of therapeutic vaccines to improve efficacy and provide convenience.

1-28. (canceled)

29. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; wherein the device delivers at least 50% of the antigenic agent to the skin of a subject after the device is applied to the skin of the subject for one hour.

30. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; wherein the device delivers at least 50% of the antigenic agent to the skin of a subject after the device is applied to the skin of the subject for one hour; and
wherein the immune response augmenting adjuvant is selected from the group consisting of aluminum phosphate gel, aluminum hydroxide, algal glucan, β-glucan, cholera toxin B subunit, heat-shock proteins (HSPs), gamma inulin, GMDP (N-acetylglucosamine-(β1-4)-N-acetylmuramyl-L-alanyl-D-glutamine), GTP-GDP, Imiquimod, ImmTher™ (DTP-GDP), Loxoribine, MPL®, MTP-PE, Murametide, Pleuran (beta-glucan), Murapalmitine, QS-21, S-28463 (4-Amino-α,α-dimethyl-1H-imidazo[4,5-c]quinoline-1-ethanol), Sclavo Peptide (IL-1β163-171 peptide), and Theramide™.

31. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein the adjuvant comprises glucosaminyl muramyl dipeptide; wherein the device delivers at least 50% of the antigenic agent to the skin of a subject after the device is applied to the skin of the subject for one hour.

32. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein the array has a skin contact area and said reservoir has an antigenic agent loading of at least about 0.2 μg/cm² of the skin contact area of said array; wherein the device delivers at least 50% of the antigenic agent to the skin of a subject after the device is applied to the skin of the subject for one hour.

33. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein said array has a skin contact area and said reservoir has an antigenic agent loading of at least about 2 μg/cm² of said skin contact area of said array; wherein the device delivers at least 50% of the antigenic agent to the skin of a subject after the device is applied to the skin of the subject for one hour.

34. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein the antigenic agent is selected from the group consisting of proteins, polysaccharides, oligosaccharides, lipoproteins, weakened or killed viruses, weakened or killed bacteria and mixtures thereof; wherein the device delivers at least 50% of the antigenic agent to the skin of a subject after the device is applied to the skin of the subject for one hour.

35. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein said antigenic agent comprises a vaccine; wherein the device delivers at least 50% of the antigenic agent to the skin of a subject after the device is applied to the skin of the subject for one hour.

36. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein said vaccine is selected from the group consisting of flu vaccines, Lyme disease vaccine, rabies vaccine, measles vaccine, mumps vaccine, chicken pox vaccine, small pox vaccine, hepatitis vaccine and diphtheria vaccine; wherein the device delivers at least 50% of the antigenic agent to the skin of a subject after the device is applied to the skin of the subject for one hour.

37. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein said array is comprised of metal and includes an adhesive backing; wherein the device delivers at least 50% of the antigenic agent to the skin of a subject after the device is applied to the skin of the subject for one hour.

38. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein said array has a skin contact area of up to about 5 cm²; wherein the device delivers at least 50% of the antigenic agent to the skin of a subject after the device is applied to the skin of the subject for one hour.

39. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein the weight ratio of adjuvant loading to antigenic agent loading in the reservoir, is in the range of about 0.5:1 to 50:1; wherein the device delivers at least 50% of the antigenic agent to the skin of a subject after the device is applied to the skin of the subject for one hour.

40. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein the weight ratio of adjuvant loading to antigenic agent loading in the reservoir, is in the range of about 1:1 to 10:1; wherein the device delivers at least 50% of the antigenic agent to the skin of a subject after the device is applied to the skin of the subject for one hour.

41. An intradermal vaccine delivery device comprising: a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, the reservoir being positioned relative to said microprojections to be in agent and adjuvant transmitting relationship with said holes; and wherein said reservoir comprises a dry solid coating on the microprojections; wherein the coating is applied to the first 150 μm of the tip of the microprojections, wherein the device delivers at least 50% of the antigenic agent to the skin of a subject after the device is applied to the skin of the subject for one hour.

42. An intradermal vaccine delivery device comprising, a microprojection array, the array having a plurality of stratum corneum piercing microprojections, said microprojections having a size which is adapted to cut holes in the stratum corneum by piercing the skin to a depth of less than about 500 μm; a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, the reservoir being positioned relative to said microprojections to be in agent and adjuvant transmitting relationship with said holes; and wherein said reservoir comprises a film laminated to said array; wherein the device delivers at least 50% of the antigenic agent to the skin of a subject after the device is applied to the skin of the subject for one hour.

43. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; and delivering said antigenic agent and said adjuvant intradermally to the mammal from said reservoir; wherein at least 50% of the antigenic agent is delivered to the skin of the mammal after the microprojection array is applied to the skin of the mammal for one hour.

44. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; wherein at least 50% of the antigenic agent is delivered to the skin of the mammal after the microprojection array is applied to the skin of the mammal for one hour; and
wherein the immune response augmenting adjuvant is selected from the group consisting of aluminum phosphate gel, aluminum hydroxide, algal glucan, β-glucan, cholera toxin B subunit, heat-shock proteins (HSPs), gamma inulin, GMDP (N-acetylglucosamine-(β1-4)-N-acetylmuramyl-L-alanyl-D-glutamine), GTP-GDP, Imiquimod, ImmTher™ (DTP-GDP), Loxoribine, MPL®, MTP-PE, Murametide, Pleuran (β-glucan), Murapalmitine, QS-21, S-28463 (4-Amino-α,α-dimethyl-1H-imidazo[4,5-c]quinoline-1-ethanol), Sclavo Peptide (IL-1 β163-171 peptide), and Theramide™.

45. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein the adjuvant comprises glucosaminyl muramyl dipeptide; wherein at least 50% of the antigenic agent is delivered to the skin of the mammal after the microprojection array is applied to the skin of the mammal for one hour.

46. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein the array has a skin contact area and said reservoir has an antigenic agent loading of at least about 0.2.mu.g/cm² of the skin contact area of said array; wherein at least 50% of the antigenic agent is delivered to the skin of the mammal after the microprojection array is applied to the skin of the mammal for one hour.

47. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein said array has a skin contact area and said reservoir has an antigenic agent loading of at least about 2 μg/cm² of said skin contact area of said array; wherein at least 50% of the antigenic agent is delivered to the skin of the mammal after the microprojection array is applied to the skin of the mammal for one hour.

48. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein the antigenic agent is selected from the group consisting of proteins, polysaccharides, oligosaccharides, lipoproteins, weakened or killed viruses, weakened or killed bacteria and mixtures thereof; wherein at least 50% of the antigenic agent is delivered to the skin of the mammal after the microprojection array is applied to the skin of the mammal for one hour.

49. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein said antigenic agent comprises a vaccine; wherein at least 50% of the antigenic agent is delivered to the skin of the mammal after the microprojection array is applied to the skin of the mammal for one hour.

50. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein said vaccine is selected from the group consisting of flu vaccines, Lyme disease vaccine, rabies vaccine, measles vaccine, mumps vaccine, chicken pox vaccine, small pox vaccine, hepatitis vaccine and diphtheria vaccine; wherein at least 50% of the antigenic agent is delivered to the skin of the mammal after the microprojection array is applied to the skin of the mammal for one hour.

51. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein said array is comprised of metal and includes an adhesive backing; wherein at least 50% of the antigenic agent is delivered to the skin of the mammal after the microprojection array is applied to the skin of the mammal for one hour.

52. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein said array has a skin contact area of up to about 5 cm²; wherein at least 50% of the antigenic agent is delivered to the skin of the mammal after the microprojection array is applied to the skin of the mammal for one hour.

53. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein the weight ratio of adjuvant loading to antigenic agent loading in the reservoir, is in the range of about 0.5:1 to 50:1; wherein at least 50% of the antigenic agent is delivered to the skin of the mammal after the microprojection array is applied to the skin of the mammal for one hour.

54. A method for vaccinating a mammal, comprising: placing a microprojection array against a skin site of the mammal, said array having a plurality of skin-piercing microprojections, said microprojections having a size which is adapted to pierce the skin to a depth of less than about 500.mu.m, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein the weight ratio of adjuvant loading to antigenic agent loading in the reservoir, is in the range of about 1:1 to 10:1; wherein at least 50% of the antigenic agent is delivered to the skin of the mammal after the microprojection array is applied to the skin of the mammal for one hour.

55. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein said reservoir comprises a dry solid coating on the microprojections, and wherein the coating is applied to the first 150 μm of the tip of the microprojections; wherein at least 50% of the antigenic agent is delivered to the skin of the mammal after the microprojection array is applied to the skin of the mammal for one hour.

56. A method for vaccinating a mammal, comprising: placing a microprojection array against a skin site of the mammal, said array having a plurality of skin-piercing microprojections, said microprojections having a size which is adapted to pierce the skin to a depth of less than about 500 μm, and a reservoir containing an antigenic agent and an immune response augmenting adjuvant, wherein the reservoir is in the form of a solid coating on the surfaces of the tips of the microprojections, and the coating is applied to the first 150 μm of the tip of the microprojections, said reservoir being positioned relative to the microprojections to be in agent and adjuvant transmitting relationship with cuts in the stratum corneum formed by the piercing microprojections; causing said microprojections to pierce the skin; delivering said antigenic agent and said adjuvant intradermally to the mammal from said reservoir; and wherein said reservoir comprises a film laminated to said array; wherein at least 50% of the antigenic agent is delivered to the skin of the mammal after the microprojection array is applied to the skin of the mammal for one hour.
